(19) United States
(12) Patent Application Publication
(10) Pub. No.: US 2026/0355374 A1
(43) Pub. Date: Nov. 12, 2026

(54) ATTACHMENT SUPPRESSION/REMOVAL METHOD

(71) Applicants: The Chugoku Electric Power Co., Inc., Hiroshima-shi, Hiroshima (JP); Sessile Research Corporation, Himeji-Shi, Hyogo (JP)

(72) Inventors: Toshiharu YANAGAWA, Hiroshima-shi, Hiroshima (JP); Yurika NISHIDA, Hiroshima-shi, Hiroshima (JP); Keiji YAMASHITA, Himeji-Shi, Hyogo (JP); Kyoko KAMIYA, Himeji-Shi, Hyogo (JP); Maki OTA, Himeji-Shi, Hyogo (JP); Shinsuke SAITO, Himeji-Shi, Hyogo (JP); Kazumi FUKUMOTO, Himeji-Shi, Hyogo (JP); Yoshio HAYASHI, Himeji-Shi, Hyogo (JP); Yuuki FUKASAWA, Himeji-Shi, Hyogo (JP)

(21) Appl. No.: 19/515,866

(22) PCT Filed: Sep. 4, 2023
(86) PCT No.: PCT/JP2023/032192

§ 371 Date: Mar. 3, 2026

(51) Int. Cl. A61L 2/084

(52) U.S. Cl. CPC A61L 2/084

(57) ABSTRACT

This attachment suppression/removal method includes irradiating a structure having a sessile organism attached thereto with light on the basis of a light irradiation amount set for every sessile organism, thereby suppressing the attachment of the sessile organism to the structure or removing the sessile organism attached to the structure. The light irradiation amount is 160 Whm⁻² or more, and the irradiance of the light irradiated on the basis of the light irradiation amount is 20 Wm⁻² or more.

### TECHNICAL FIELD

The present invention relates to an attachment suppression and removal method.

### BACKGROUND ART

In recent years, technology has been known for preventing the attachment of organisms such as barnacles to structures such as water quality meters immersed in water, or the seawater system equipment of thermal power plants, nuclear power plants, etc. For example, a method has been known in Patent Document 1 that prevents the attachment of sessile organisms to structures, by irradiating light including a wavelength of 409 to 412 nm onto structures in water.

### CITATION LIST

### Patent Documents

Patent Document 1: Japanese Unexamined Patent Application, Publication No. 2016-188570

### DISCLOSURE OF THE INVENTION

### Problems to be Solved by the Invention

The method disclosed in Patent Document 1 suppresses and prevents new attachments to a foundation, and there has been room for improvement in the point of more reliably suppressing attachment and in the case of decreasing or removing many already attached and growing on the foundation.

The present invention has an object of providing an attachment suppression and removal method which can reliably perform suppression of attachment of sessile organisms to a structure, or removal of sessile organisms from a structure.

### Means for Solving the Problems

(1) The present invention relates to an attachment suppression and removal method for a structure to which a sessile organism attaches, the method including performing suppression of attachment of the sessile organism to a structure or removal of the sessile organism attached to the structure, by irradiating light onto the structure, based on a light irradiation quantity set for every kind of sessile organism.
(2) In the attachment suppression and removal method as described in (1), the light irradiation quantity is 160 Whm⁻² or more.
(3) In the attachment suppression and removal method as described in (1), the light irradiation quantity is 480 Whm⁻² or more.
(4) In the attachment suppression and removal method as described in (1), the light irradiation quantity is 1920 Whm⁻² or more.
(5) In the attachment suppression and removal method as described in (1), the light irradiation quantity is 4800 Whm⁻² or more.
(6) In the attachment suppression and removal method as described in any one of (1) to (5), irradiance of light irradiated based on the light irradiation quantity is 20 Wm⁻² or more.
(7) In the attachment suppression and removal method as described in any one of (1) to (6), irradiance of light irradiated based on the light irradiation quantity is 24000 Wm-2 or more.
(8) In the attachment suppression and removal method as described in any one of (1) to (7), light irradiated based on the light irradiation quantity has a peak in a wavelength region of 400 to 490 nm.
(9) In the attachment suppression and removal method as described in any one of (1) to (7), light irradiated based on the light irradiation quantity has a peak in a wavelength region of 400 to 430 nm.

### Effects of the Invention

According to the present invention, it is possible to reliably perform suppression of attachment of sessile organisms to a structure, or removal of sessile organisms from a structure.

### BRIEF DESCRIPTION OF THE DRAWINGS

FIG. 1 is a view showing the temporal state change of Amphibalanus amphitrite cypris larva when continuously irradiating LED light (wavelength peak: 405 nm) at 200 Wm⁻² irradiance;

FIG. 2 is a view showing the temporal state change of Amphibalanus amphitrite cypris larva in a control section of the test in which the results shown in FIG. 1 were obtained; and

FIG. 3 is a view showing a relationship between juvenile barnacle rate and irradiance in the case of continuously irradiating LED light (wavelength peak: 405 nm) at each irradiance for 96 hours.

### PREFERRED MODE FOR CARRYING OUT THE INVENTION

Hereinafter, embodiments of the present invention will be described. It should be noted that the present invention is not limited to the following embodiments, and modifications are possible where appropriate.

### <Attachment Suppression/Removal Method>

An attachment suppression/removal method according to the present embodiment is a method relating to structures to which sessile organisms attach for performing suppression of attachment of sessile organisms to a structure (hereinafter referred to as attachment suppression) or removal of sessile organisms attached to a structure (hereinafter referred to as sessile organism removal), by irradiating light onto a structure based on light exposure dosage set for every sessile organism.

As the structure to which sessile organisms attach, a structure in water (including structures that are permanently or temporarily wetted by a liquid such as water or sweat) or structures installed in air, for example, seawater system equipment in power generation facilities such as thermal power plants and nuclear power plants, and inspection windows for performing inspection of these facilities can be exemplified. Other than this, the structure may be a water quality meter, underwater camera device, water intake pipe, rotary screen, bar screen, drum screen, mussel filter, net screen, water intake pump, circulating water pump, circulating water pipe, heat exchanger, steam condenser, bearing cooling water cooler, lubricating oil cooler, LNG vaporizer, generator, drainage pipe, water turbine, impeller, valve, spindle, water pipeline, filtration tank, membrane, dam, ship, ship hull at shipyard, ballast water tank, ballast water intake/discharge pipes, pumps, aquaculture equipment, fisheries research facility, fishery equipment, aquarium, water tank for seafood breeding tank, plumbing, pumps, cooling tower water tank and radiating plates, scrubber water tank, etc. In addition, for example, it may be the floor or the like of a bathroom, pool, shower room or the like. In addition, it may be indoor seats or in a vehicle that are temporarily wetted by sweat. Furthermore, it may be the exterior wall or interior wall of a building, window exterior surface or interior surface, desk, chair, hospital operating room, patient room, etc. In addition, structure referenced herein may be a living body. For example, it may be the shell or molluscous part of oysters, fish, protozoa parasitizing fish, plankton, algae, microorganisms, parts of biological tissues, skin, teeth, cells and their secretions, and the surfaces of these can be the target of attaching by sessile organisms. For example, air bubbles in water can be the structure to which sessile organisms attach. In other words, the air bubble surface in water or liquid can be the target of several attaching microorganisms accumulating and attaching. In addition, the surface of a pack container, inner faces thereof and the foodstuff inside can be the target of accumulation and adherence of several attaching microorganisms. In addition, foam, cream, jelly, gel and mist can also be the target of accumulation and adherence of attaching microorganisms.

Sessile organism is an organism having a property of attaching to an underwater structure (including structures that are permanently or temporarily wetted by a liquid such as water or sweat) or the surface of a structure in air. The sessile organism may be an organism, for example, that floats in the sea during the early larval stage, and when becoming sessile larva, attaches to a suitable target and metamorphosizes into an adult. As the sessile organism, for example, animals belonging to mussels, barnacles, hydrozoa, sponges, epiphytic generation of jellyfish, tubiculous polychaeta, bryozoa, amphipods, etc.; and microorganisms belonging to bacteria, viruses, eumycetes, cyanophyta, protozoa, etc. can be exemplified. In addition, biofilms which are structures formed by microorganisms are also included in the sessile organisms of the present disclosure. Biofilms are usually membrane form, and include extracellular polymeric substances (EPS) such as polysaccharides secreted by microorganisms. In addition, the remains, excretions, feces, dwelling tubes, gene fragments, organic matter, inorganic particles of microorganisms, etc. may be included in biofilms. It should be noted that, in the case of a very thin biofilm being formed on a seat surface or floor surface indoors or in a vehicle, foodstuff, a pack container surface, inner face, internal article, biont, algae, tissue, cells, skin, teeth or the like, this biofilm can be regarded as a sessile organism.

The light irradiation quantity is the total amount of light irradiated onto a structure for attachment suppression or sessile organism removal. The light irradiation quantity may be set according to the tolerance to light of the sessile organism, for example. In this case, the light irradiation quantity may be set to the same value for a plurality of sessile organisms having different tolerances to light, for example, or may each be set to different values. In the case of setting to the same value, an irradiation quantity effective for attachment suppression or sessile organism removal of the sessile organism having the highest tolerance to light may be set. By irradiating light onto a structure based on the irradiation quantity of light set for every sessile organism, it is possible to reliably perform attachment suppression or sessile organism removal.

The light irradiation quantity set in the present embodiment is preferably 160 Whm⁻² or more, more preferably 200 Whm⁻² or more, more preferably 400 Whm⁻² or more, more preferably 480 Whm⁻² or more, more preferably 1920 Whm⁻² or more, more preferably 2400 Whm⁻² or more, more preferably 4800 Whm⁻² or more, and particularly preferably 9600 Whm⁻² or more. By irradiating light of the irradiation quantity 200 Whm⁻² or more onto a sessile organism such as barnacles having a relatively high tolerance to light, it is possible to cause the population which is incapable of swimming (i.e. incapable of attaching to structure) to increase. In addition, by irradiating light of an irradiation quantity of 400 Whm⁻² or more onto a sessile organism such as barnacles, it is possible to increase the population in a state designated as unresponsive such as a thoracic appendage protruding state. In addition, it is possible to reduce the colony appearance ratio of microorganisms such as bacteria by setting the light irradiation quantity to 480 Whm⁻² or more. In other words, it is possible to suppress the generation of a biofilm formed from microorganisms as a substrate that promotes attachment of a large-scale sessile organism such as the larva of barnacles, mussels or the like to a structure. Consequently, it is possible to suppress attachment of a sessile organism having relatively high tolerance to light to a structure. By setting the light irradiation quantity to 1920 Whm⁻² or more, it is possible to further reduce the population of barnacles attaching to a structure, and by setting the light irradiation quantity to 2400 Whm⁻² or more, it is possible to suppress attachment of barnacles to a structure. In addition, by setting the light irradiation quantity to 4800 Whm⁻² or more, it is possible to remove barnacles that have already attached to a structure, and by setting the light irradiation quantity to 9600 Whm⁻² or more, it is possible to more reliably remove barnacles that have already attached to a structure.

The irradiance of light irradiated based on the light irradiation quantity set in the present disclosure is preferably 20 Wm⁻² or more, more preferably 100 Wm⁻² or more, and even more preferably 24000 Wm⁻² or more. By setting the light irradiance to 20 Wm⁻² or more and irradiating the light irradiation quantity set for every sessile organism, it is possible to reliably suppress attachment of sessile organisms to a structure. In addition, by setting the light irradiation quantity to 24000 Wm⁻² or more, it is possible to perform attachment suppression or sessile organism removal in a shorter time. Consequently, it is possible to perform attachment suppression or sessile organism removal in a relatively short time also in the case of intermittently irradiating light while moving.

The light irradiated onto the structure preferably has a peak in the wavelength region of 400 to 430 nm. Although ultraviolet rays having a wavelength of 380 nm or less have an attachment suppression effect for sessile organisms, there is a problem in that the transmittance in seawater is low. By setting the wavelength of irradiated light to the above-mentioned range, the preferred attachment suppression effect and sessile organism removal effect are obtained. The wavelength of irradiated light may have a peak in a wavelength region outside the above, within a range not inhibiting the effects of the present invention. For example, the wavelength of irradiated light may have a peak in the wavelength region of 430 to 490 nm.

In addition, with the attachment suppression or sessile organism removal of the present embodiment, light may be continuously irradiated (hereinafter referred to as continuous irradiation) or may be intermittently irradiated (hereinafter referred to as intermittent irradiation) onto a structure.

### EXAMPLES

Hereinafter, the contents of the present invention will be described in further detail based on the Examples. The contents of the present invention are not limited to the descriptions of the following Examples.

### (Influence of Light Irradiation on Barnacles Confirmation Test)

Using cypris larva of Amphibalanus amphitrite (hereinafter referred to as cypris larva), the attachment suppression effect on organisms by LED light was confirmed. It should be noted that cypris larva is larva in the adhesion period, reached by nauplius larva in the floating period repeating molting, and growing to be attachable to a structure. Juvenile barnacles described later are barnacles reached by cypris larva attaching to a structure such as a test container, and undergoing metamorphosis.

To a test container (cylinder of 6 cm diameter×6 cm height), 70 ml of test seawater (seawater filtered through 0.45 μm pore-size membrane), and cypris larva (population: 44+/−12) were added. As a test section, LED light (peak wavelength: 405 nm) was irradiated through a glass cover to this test container. As the light source of the LED light, a UFL-501-08 UV-UT (manufactured by U-Technology Co., Ltd.) was used. In order to prevent a temperature rise of the test seawater or the like due to irradiation of LED light, in the test, the bottom outer side of the test container was immersed in tap water. As test sections, tests were performed under the five conditions of irradiance of 20 Wm⁻², 50 Wm⁻², 100 Wm⁻², 150 Wm⁻² and 200 Wm⁻². In the tests, continuous irradiation was performed for 96 hours under each irradiance condition. In the tests, the state of the cypris larva in the test container was observed twice per day to obtain the appearance ratio of cypris larva in each state. The test container was covered with aluminum foil, and the portion establishing substantially constant dark conditions without irradiating LED light was set as a control section. It should be noted that irradiance was measured and adjusted through the lid of the test container using an Ophir StarLite PD300Rm-8W radiometer. In all of the following tests, the irradiance was measured and adjusted using this radiometer.

### (Test Results)

### <Cypris Larva State Change Under Light Irradiation of 200 Wm⁻² Irradiance>

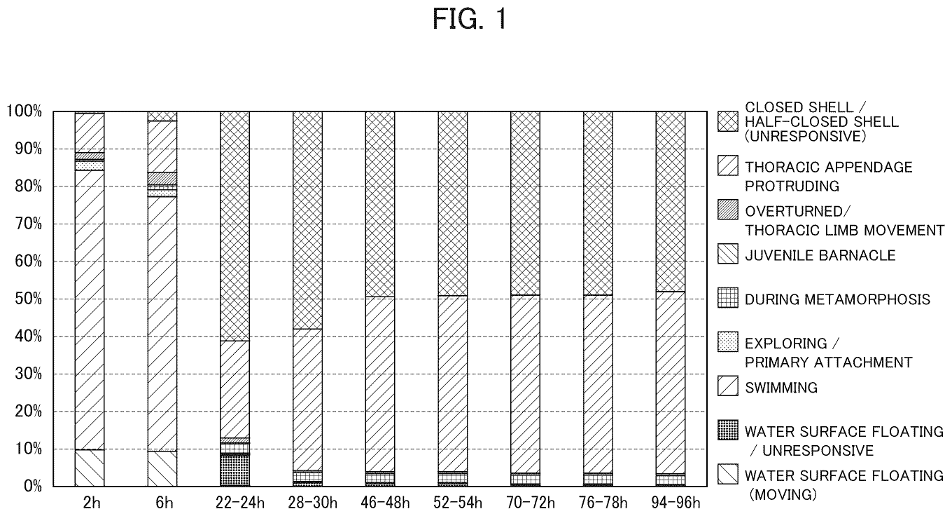

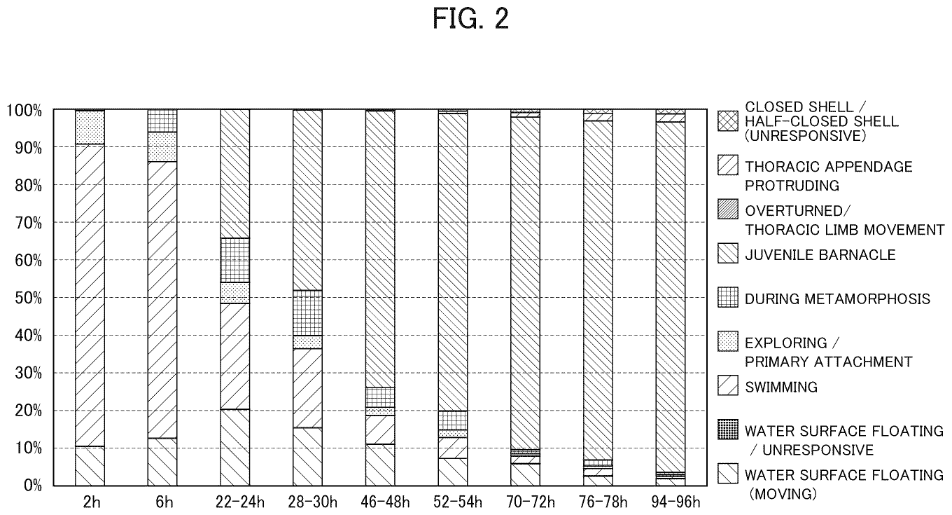

FIG. 1 is a graph showing the temporal state change of cypris larva in the test section of 200 Wm⁻² irradiance. FIG. 2 is a graph showing the temporal state change of cypris larva in the control section relative to the test section of 200 Wm⁻² irradiance. The horizontal axis in FIGS. 1 and 2 shows the elapsed time from the start of testing, and the vertical axis shows the appearance ratio of cypris larva in each state.

As shown in FIG. 2, in the control section, 80% or more of the individuals of cypris larva were swimming after 2 hours from the test start, and when 6 hours elapsed, they gradually attached to the test container and started metamorphosis, 30% or more of the individuals becomes juvenile barnacles after 24 hours, and the appearance ratio of juvenile barnacles after 96 hours reached 90% or more. In other words, it was confirmed that 90% or more barnacles attached to the test container.

On the other hand, as shown in FIG. 1, in the case of the test section performed under irradiation of 200 Wm⁻² irradiance, 80% or more of the individuals of cypris larva entered a thoracic appendage protruding state 2 hours from the start of irradiation, 95% or more of individuals were unresponsive in a closed shell or half-closed shell state, thoracic appendage protruding state, or unresponsive state in a state floating on the water surface, after 24 hours with the irradiation quantity of 4800 Whm⁻². From this result, it is speculated that a decrease in barnacle attachment began with the irradiation quantity of 400 Whm⁻² or more, and was almost completely suppressed with 4800 Whm⁻².

### <Juvenile Barnacle Rate Under Light Irradiation of Each Irradiance>

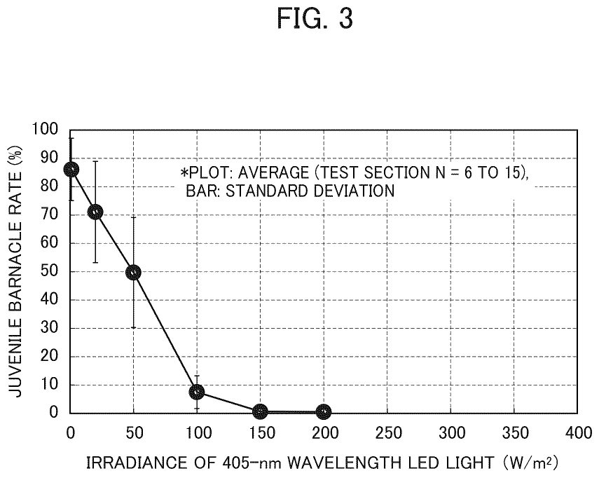

FIG. 3 is a graph showing the relationship between juvenile barnacle rate and each irradiation condition in the case of continuous irradiation of LED light for 96 hours at each irradiance. The horizontal axis in FIG. 3 shows the irradiance (Wm⁻²), and the vertical axis shows the juvenile barnacle rate (%). It should be noted that juvenile barnacle rate is a proportion of juvenile barnacles among the Amphibalanus amphitrite existing in each test container. By evaluating the barnacles having metamorphosized from cypris larva to juvenile barnacle, it is possible to evaluate the attachment suppression effect for barnacles to a structure.

As shown in FIG. 3, in the control section, the juvenile barnacle rate after 96 hours was about 85% or more. In other words, it could be confirmed that 85% or more of barnacles had attached to the bottom surface or lateral surfaces of the petri dish. In contrast, in the test section of 150 Wm⁻² irradiance, the juvenile barnacle rate after 96 hours was 0.6+/−1.4%, and the verified very few juvenile barnacles also were all confirmed to have died. In other words, the growth of cypris larva was completely suppressed with 150 Wm⁻² irradiance, and it was confirmed that attachment to a structure could be prevented. In addition, the juvenile barnacle rate of the test section with 20 Wm⁻² irradiance was also reduced to about 70%. From this result, it is speculated that the attached amount of sessile organisms to a structure could be reduced by irradiating with the irradiation quantity of 1920 Whm⁻².

<Swimming Incapable Rate of Cypris Larva after 2 Hours and 6 Hours from Irradiation Start>

Table 1 shows the appearance ratio of swimming incapable individuals of cypris larva (hereinafter referred to as swimming incapable rate) after 2 hours and 6 hours from the start of LED light irradiation at each irradiance, and the irradiation quantity (Whm⁻²). Swimming incapable individual referenced herein indicates the individuals from combining the overturned/thoracic limb movement individuals (individuals moving thoracic appendage in overturned state), thoracic appendage protruding individuals and closed shell/half-closed shell (unresponsive) individuals. It should be noted that N number in the table is the number of times testing (same in following tables).

TABLE 1

| | | 2-hr irradiation time | | 6-hr irradiation time | |
|---|---|---|---|---|---|
| | | Irradiation | Swimming | Irradiation | Swimming |
| Irradiance | | quantity | incapable | quantity | incapable |
| (Wm⁻²⁾ | N number | (Whm⁻²) | rate (%) | (Whm⁻²) | rate (%) |
| 0 (control section) | 97 | 0 | 0 | 0 | 0 |
| 20 | 15 | 40 | 0 | 120 | 0 |
| 50 | 6 | 100 | 0 | 300 | 1.8 ± 1.6 |
| 100 | 15 | 200 | 5.8 ± 10.1 | 600 | 9.4 ± 14.8 |
| 150 | 6 | 300 | 6.7 ± 3.1 | 900 | 10.7 ± 5.3 |
| 200 | 15 | 400 | 13.2 ± 10.1 | 1200 | 19.5 ± 18.2 |

As shown in Table 1, it was confirmed that swimming incapable individuals of cypris larva did not appear with the irradiation quantity of 100 Whm⁻². On the other hand, it was confirmed that the swimming incapable rate reached 5.8+/−10.1% with the irradiation quantity of 200 Whm⁻², reached about 10% with 900 Whm⁻², and reached about 20% with 1200 Whm⁻². From this result, it is speculated that the biological activity of barnacle adhesion period larva started to decline with the LED light irradiation quantity of 200 Whm⁻² or more. In other words, it is speculated that barnacle attachment started to decrease with the irradiation quantity of 200 Whm⁻² or more.

### <Distribution of Barnacle Attachment Positions>

Table 2 is a table showing the positional distribution of barnacles attaching to a petri dish in the test section of 20 Wm⁻² irradiance, and the control section thereof. Table 3 is a table showing the positional distribution of barnacles attaching to a petri dish in the test section of 50 Wm⁻² irradiance, and the control section thereof.

TABLE 2

| Irradiance | 0 | |
|---|---|---|
| (Wm⁻²⁾) | (control section) | 20 |
| N number | 22 | 15 |
| Attachment ratio to | 18.8 ± 7.3 | 46.0 ± 12.4 |
| lateral surface (%) | | |
| Attachment ratio to | 81.2 ± 7.3 | 54.0 ± 12.4 |
| bottom surface (%) | | |

TABLE 3

| Irradiance | 0 | |
|---|---|---|
| (Wm⁻²⁾) | (control section) | 50 |
| N number | 10 | 6 |
| Attachment ratio to | 20.8 ± 8.6 | 44.7 ± 19.3 |
| lateral surface (%) | | |
| Attachment ratio to | 79.2 ± 8.6 | 55.3 ± 19.3 |
| bottom surface (%) | | |

As shown in Table 2 and Table 3, at both conditions of 20 Wm⁻² irradiance and 50 Wm⁻² irradiance, about 80% of individuals attached to the bottom surface of the petri dish and about 20% of individuals attached to a lateral surface of the petri dish in the control section. On the other hand, the difference in attachment ratio to the bottom surface and lateral surface of the petri dish in the test section could be confirmed to be small. As a result of performing multiple comparison tests (Bonferroni method) for lateral surface attachment rate in the control section and lateral surface attachment rate in the test section, it was confirmed that the lateral surface attachment rate to the petri dish in the test section increased significantly (p<0.01). From this result, it is speculated that there is a tendency for the cypris larva to avoid locations where LED light (wavelength peak: 405 nm) is directly irradiated, even when attached.

### (Influence of Light Irradiation on Hydrozoa Confirmation Test)

The influence of light irradiation on hydrozoa was confirmed using a hydra stem segment of Coryne pusilla which is a hydrozoan. It should be noted that the hydra stem segments were prepared by isolating the polyps from a Coryne pusilla colony, and cutting off the hydra roots and hydra flower.

To a test container (petri dish of 6-cm diameter×6-cm height), 70 ml of test seawater (seawater filtered through 0.45 μm pore-size membrane) and hydra stem segments (count: 12 segments) were added. As test sections, LED light (peak wavelength: 405 nm) was continuously irradiated with the respective irradiances shown in Table 4 below, through a glass cover to this test container. The test container was covered with aluminum foil, and the portion establishing substantially constant dark conditions without irradiating LED light was defined as the control section. The test was performed for 96 hours to compare the regeneration rate of hydra stem fragments and regenerated hydra flower number between the control section and each test section.

TABLE 4

| Irradiance | | | |
|---|---|---|---|
| (Wm⁻²) | 0 | 100 | 200 |
| N number | 12 | 6 | 6 |
| Regeneration rate | 100.0 ± 0.0 | 0.0 ± 0.0 | 0.0 ± 0.0 |
| average +/− SD after | | | |
| 96 hr (%) | | | |
| Total average +/− SD | 16.4 ± 2.5 | 0.0 ± 0.0 | 0.0 ± 0.0 |
| of hydra flower | | | |
| number after 96 hr | | | |
| (%) | | | |

### (Test Results)

Table 4 shows the regeneration rate of hydra stem segments and regenerated hydra flower number after 96 hours in the test sections with irradiance of 100 Wm⁻² and 200 Wm⁻² and the control section. As shown in Table 4, in the case of continuously irradiating for 96 hours with the two irradiances of 100 Wm⁻² and 200 Wm⁻², it was confirmed that the regeneration rate of hydra stem segments and regenerated hydra flower number were 0%. In other words, by irradiating the irradiation quantity of at least 9600 Whm⁻² or more, it could be confirmed that colony formation of marine hydrozoans could be completely prevented.

### (Comparative Test of Influence on Sessile Organism by Irradiance Difference)

Using cypris larva of Amphibalanus amphitrite, the influences on the cypris larva when irradiating the same irradiation quantity at different irradiance were compared.

To a test container (24-well microplate), 2 ml of test seawater (seawater filtered through 0.45 μm pore-size membrane), and cypris larva of Amphibalanus amphitrite (population: 16+/−4) were added. As the test section, LED light (peak wavelength: 405 nm) was continuously irradiated under the irradiation conditions shown in Table 5. As the light source of LED light, a UFL-501-08 UV-UT (manufactured by U-Technology Co., Ltd.) was used. In order to prevent a temperature rise of the test seawater or the like due to irradiation of LED light, in the test, the bottom outer side of the test container was immersed in tap water. The test container was covered with aluminum foil, and the portion establishing substantially constant dark conditions without irradiating LED light was set as the control section. The swimming incapable rate of cypris larva was confirmed by the tests.

TABLE 5

| Irradiance (Wm⁻²) | 600 | 6000 |
|---|---|---|
| Irradiation time (h) | 8 | 0.8 |
| N number | 60 | 55 |
| Irradiation quantity | 4800 | 4800 |
| (Whm⁻²) | | |
| Swimming incapable | 99.3 ± 1.9 | 99.8 ± 1.2 |
| rate average +/− SD | | |
| (%) | | |

### (Test Results)

Table 4 shows the swimming incapable rates in the test sections at the irradiation conditions of 600 Wm⁻² irradiance and 8-hr irradiation time, and at the irradiation conditions of 6000 Wm⁻² irradiance and 0.8-hr irradiation time. As shown in Table 5, the swimming incapable rate at the irradiation conditions of 600 Wm⁻² irradiance and 8-hr irradiation time, and at the irradiation conditions of 6000 Wm⁻² irradiance and 0.8-hr irradiation time were both 99% or more. As a result of performing a significance test in multiple comparison test (Tukey HSD method) by statistical software R on the swimming incapable rate of these two irradiation conditions, a significant difference was not recognized between them. In addition, as a result of a TOSTER equivalency test, the two were evaluated as statistically equal. From this result, the action of attachment suppression and organism removal was confirmed to be substantially proportional to the product of irradiance and irradiation time, i.e. irradiation quantity. In addition, by irradiating an irradiation quantity of 4800 Whm⁻², it is speculated that attachment of sessile organisms to a structure can be prevented.

### (Attachment Removal Effect Confirmation Test)

The attachment removal effect from light irradiation onto attached individuals of barnacles attached to a structure such as a test container was confirmed.

First, 8 ml of test seawater for attachment (seawater filtered through 0.45 μm mesh-size membrane) and cypris larva of (population: about 30) were added to a test container (6-well microplate), and left to stand for 1 to 2 days to allow the cypris larva to attach to the bottom surface of the test container. Subsequently, the unattached cypris larva were removed with the test seawater for attachment in the test container, and 8 ml of test seawater for breeding (seawater filtered through 0.45 μm mesh-size cartridge filter) was added to the test container. To the juvenile barnacles attached to the test container, Chaetoceros calcitrans were given as feed to allow to grow during each breeding period. The breeding periods were set as 1 week, 2 weeks, 3 weeks or 4 weeks. After each breeding period had elapsed, among the grown barnacle attached individuals, particularly the individuals growing to large scale and performing leg movements actively were set as targets, the bottom plate diameter was measured, and LED light (wavelength peak: 405 nm or 460 nm) was irradiated with 24000 Wm⁻² irradiance. During LED light irradiation, the state of barnacle attached individuals was observed at every fixed time. As the light source of the LED light, a UFL-501-08 UV-UT (manufactured by U-Technology Co., Ltd.) was used.

### (Test Results)

Table 6 shows the abundance ratio in each state of barnacle attached individuals when irradiating LED light of 405-nm peak wavelength onto barnacle attached individuals with a breeding period of 1 week. Table 7 shows the abundance ratio in each state of barnacle attached individuals when irradiating LED light of 405-nm peak wavelength onto barnacle attached individuals with a breeding period of 2 weeks. Table 8 shows the abundance ratio in each state of barnacle attached individuals when irradiating LED light of 405-nm peak wavelength onto barnacle attached individuals with a breeding period of 3 weeks. Table 9 shows the abundance ratio in each state of barnacle attached individuals when irradiating LED light of 405-nm peak wavelength onto barnacle attached individuals with a breeding period of 4 weeks. Table 10 shows the abundance ratio in each state of barnacle attached individuals when irradiating LED light of 460-nm peak wavelength onto barnacle attached individuals with a breeding period of 2 weeks. It should be noted that the bottom plate diameter of the barnacle attached individuals serving as irradiation targets were 1.0 to 2.0 in individuals with a breeding period of 1 week, 2.0 to 4.1 mm for individuals of 2 weeks, 2.5 to 5.1 mm for individuals of 3 weeks, and 3.1 to 5.2 mm for individuals of 4 weeks.

TABLE 6

| | | | | | |
|---|---|---|---|---|---|
| Irradiation | Elapsed time (h) | 0.2 | 0.4 | 0.6 | 0.8 |
| conditions | Irradiation quantity (Whm⁻²) | 4800 | 9600 | 14400 | 19200 |
| Abundance | Continuous cirrus movement | 0 | 0 | 0 | 0 |
| ratio in each | Intermittent cirrus movement | 0 | 0 | 0 | 0 |
| state | Lid opening and closing, or | 0 | 0 | 0 | 0 |
| (%) | moved front/back while lid | | | | |
| | closed | | | | |
| | Moving front/back while | 7.0 | 0 | 0 | 0 |
| | lid half opened or immediate | | | | |
| | closing reaction when | | | | |
| | making contact | | | | |
| | Near death | 29.8 | 10.5 | 5.3 | 0 |
| | Unresponsive (dead) | 63.2 | 89.5 | 94.7 | 100 |

TABLE 7

| | | | | | |
|---|---|---|---|---|---|
| Irradiation | Elapsed time (h) | 0.1 | 0.2 | 0.4 | 0.6 |
| conditions | Irradiation quantity (Whm⁻²) | 2400 | 4800 | 9600 | 14400 |
| Abundance | Continuous cirrus movement | 0 | 0 | 0 | 0 |
| ratio in each | Intermittent cirrus movement | 0 | 0 | 0 | 0 |
| state | Lid opening and closing, or | 0 | 0 | 0 | 0 |
| (%) | moved front/back while lid | | | | |
| | closed | | | | |
| | Moving front/back while | 30.8 | 10.3 | 0 | 0 |
| | lid half opened or immediate | | | | |
| | closing reaction when | | | | |
| | making contact | | | | |
| | Near death | 12.8 | 17.9 | 7.7 | 0 |
| | Unresponsive (dead) | 56.4 | 71.8 | 92.3 | 100 |

TABLE 8

| | | | | | |
|---|---|---|---|---|---|
| Irradiation | Elapsed time (h) | 0.2 | 0.4 | 0.6 | 0.8 |
| conditions | Irradiation quantity (Whm⁻²) | 4800 | 9600 | 14400 | 19200 |
| Abundance | Continuous cirrus movement | 0 | 0 | 0 | 0 |
| ratio in each | Intermittent cirrus movement | 4.5 | 0 | 0 | 0 |
| state | Lid opening and closing, or | 4.5 | 0 | 0 | 0 |
| (%) | moved front/back while lid | | | | |
| | closed | | | | |
| | Moving front/back while | 22.7 | 9.1 | 0 | 0 |
| | lid half opened or immediate | | | | |
| | closing reaction when | | | | |
| | making contact | | | | |
| | Near death | 4.5 | 18.2 | 13.6 | 0 |
| | Unresponsive (dead) | 63.6 | 72.7 | 86.4 | 100 |

TABLE 9

| | | | | |
|---|---|---|---|---|
| Irradiation | Elapsed time (h) | 0.1 | 0.2 | 0.4 |
| conditions | Irradiation quantity (Whm⁻²) | 2400 | 4800 | 9600 |
| Abundance | Continuous cirrus movement | 0 | 0 | 0 |
| ratio in each | Intermittent cirrus movement | 0 | 0 | 0 |
| state | Lid opening and closing, or | 0 | 0 | 0 |
| (%) | moved front/back while lid | | | |
| | closed | | | |
| | Moving front/back while lid | 0.0 | 0 | 0 |
| | half opened or immediate | | | |
| | closing reaction when | | | |
| | making contact | | | |
| | Near death | 52.9 | 0 | 0 |
| | Unresponsive (dead) | 47.1 | 0 | 0 |

TABLE 10

| | | | | | | |
|---|---|---|---|---|---|---|
| Irradiation | Elapsed time (h) | 0.2 | 0.4 | 0.6 | 1.6 | 1.8 |
| conditions | Irradiation quantity (Whm⁻²) | 4800 | 9600 | 14400 | 38400 | 43200 |
| Abundance | Continuous cirrus movement | 57.1 | 28.6 | 0 | 0 | 0 |
| ratio in each | Intermittent cirrus movement | 14.3 | 42.9 | 42.9 | 14.3 | 0 |
| state | Lid opening and closing, or | 0 | 0 | 28.6 | 28.6 | 14.3 |
| (%) | moved front/back while lid | | | | | |
| | closed | | | | | |
| | Moving front/back while lid half | 28.6 | 28.6 | 28.6 | 14.3 | 42.9 |
| | opened or immediate closing | | | | | |
| | reaction when making contact | | | | | |
| | Near death | 0 | 0 | 0 | 42.9 | 28.6 |
| | Unresponsive (dead) | 0 | 0 | 0 | 0 | 14.3 |

As shown in Table 10, when irradiating LED light of 460-nm peak wavelength at 24000 Wm⁻², it could be confirmed that about half of the barnacle attached individuals were near death so long as being an irradiation quantity of 38400 Whm⁻² or more. On the other hand, as shown in Tables 6 to 9, when irradiating LED light of 405-nm peak wavelength at 24000 Wm⁻² irradiance, it was confirmed that for individuals bred and grown for 1 to 4 weeks after attachment, 90 to 100% of individuals were dead or near death within 12 to 24 minutes. In addition, individuals in an unresponsive state were seen as quickly as 2 minutes after irradiation (i.e. irradiation quantity of 800 Whm⁻²). In addition, a rise in tolerance to LED light in barnacle attached individuals as growing was not observed. In other words, so long as the irradiation quantity of LED light is 4800 Whm⁻² or more, it could be confirmed that a lethal action was exerted on the barnacle attached individuals, and were removable from the structure. Based on this, since sessile organisms including sessile organisms already on the structure can be removed by irradiating light of 4800 Whm⁻² or more irradiation quantity in a predetermined frequency such as once per month, it is possible to perform more efficient and reliable attachment suppression and sessile organism removal.

### (Influence of Light Irradiation on Bacteria Confirmation Test)

The influence of light irradiation on bacterial growth was confirmed by smearing marine bacteria onto an agar medium, followed by counting the number of colonies appearing after irradiation of LED light.

Onto the agar medium surface in a petri dish (9-cm diameter×1.5-cm height), 0.1 ml of seawater containing marine bacteria (all types of marine bacteria in sea water in barnacle breeding aquarium) was dropped and smeared, and half of the smeared agar medium was covered with aluminum foil to establish a control section. Then, LED light (peak wavelength: 405 nm) was irradiated under conditions of the respective irradiances and irradiation times shown in Table 11 and 12 from above by stacking one or two petri dishes, and the colony numbers in the control section and test section on each agar medium surface after about 24 hours from the start of irradiation were counted, and the appearance ratio of the colony number in the test section relative to the colony number in the control section was calculated.

As the agar medium, powdered culture medium (Marine Agar 2216, manufactured by Difco) was added to 1 L of distilled water, and after heating to dissolve and sterilize, about 20 ml was dispensed to the petri dish, left to stand for 30 to 60 minutes, and used as agar solid growth medium. As the seawater smeared on the agar medium, seawater in a barnacle breeding tank that had been filtered using a 0.45 μm pore-size membrane and diluted 10 times was used.

TABLE 11

| | | | | Irradiation section |
|---|---|---|---|---|
| | Irradiation | Irradiation | Bacterial colony | colony number/ |
| Irradiance | time | quantity | appearance | control section |
| (Wm⁻²) | (h) | (Whm⁻²) | ratio (%) | colony number |
| 20 | 6 | 120 | 90.14 | 128/142 |
| 20 | 24 | 480 | 68.13 | 109/160 |
| 200 | 6 | 1200 | 5.6 | 8/143 |

TABLE 12

| | | | | Bacterial | Irradiation |
|---|---|---|---|---|---|
| | | Irradi- | Irradi- | colony | section |
| Arrange- | Irradi- | ation | ation | appear- | colony number/ |
| ment of | ance | time | quantity | ance | control section |
| container | (Wm⁻²) | (h) | (Whm⁻²) | ratio (%) | colony number |
| Lower stage | 96 | 8 | 768 | 92.6 | 63/68 |
| Lower stage | 144 | 8 | 1152 | 77.6 | 52/67 |
| Lower stage | 192 | 8 | 1536 | 11.1 | 9/81 |
| Upper stage | 200 | 8 | 1600 | 1.6 | 1/62 |
| Upper stage | 300 | 8 | 2400 | 0 | 0/64 |
| Upper stage | 400 | 8 | 3200 | 0 | 0/63 |

### (Test Results)

Table 11 shows the appearance ratio of the colony number in the test sections relative to the colony number in the control section (hereinafter referred to as bacterial colony appearance ratio) when irradiating LED light under conditions of 120 Whm⁻² irradiation quantity, 480 Whm⁻² irradiation quantity, and 1200 Whm⁻² irradiation quantity. Table 12 shows the bacterial colony appearance ratio when irradiating LED light under conditions of 768 Whm⁻² irradiation quantity, 1152 Whm⁻² irradiation quantity, and 1536 Whm⁻² irradiation quantity onto the agar medium on the lower stage when arranging two petri dishes to be stacked, and the bacterial colony appearance ratio when irradiating 1600 Whm⁻² irradiation quantity, 2400 Whm⁻² irradiation quantity, and 3200 Whm⁻² irradiation quantity onto the agar medium in the upper stage.

As shown in Table 11 and Table 12, it could be confirmed that, by irradiating the irradiation quantity of 480 Whm⁻², the bacterial colony appearance ratio was curbed to less than 70%, by irradiating the irradiation quantity of 1200 Whm⁻², the bacterial colony appearance ratio was curbed to less than 6%, and by irradiating the irradiation quantity of 2400 Whm⁻² or more, the generation of bacterial colonies could be prevented. It could be confirmed that a high removal effect was exhibited with a fixed irradiance or more on the agar medium on the lower stage where LED light was not directly irradiated.

### (Influence of Light Irradiation on Microorganisms in Air Confirmation Test 1)

By culturing microorganisms dropped onto LB medium, and counting the colony number appearing after the irradiation of LED light, the influence from light irradiation on the microorganisms attaching to a structure in air was confirmed.

LB medium was prepared by autoclave treatment of a mixture made by adding and mixing 5 g of tryptone, 2.5 g of yeast extract, 5 g of NaCl, 7.5 g of agar, and 480 ml of distilled water to a medium bottle, for 20 minutes at 121 °C, and dispensing 20 ml of this onto a petri dish and allowing to solidify at room temperature. In a state with the lids of the three petri dishes in which this LB medium is placed left open, they were placed on a counter about 90 cm height above the floor within a room in the vicinity of the exit to outdoors, and left for 8 hours. The lids were closed after 8 hours, the three petri dishes were respectively labeled Samples 1 to 3, LED light (peak wavelength: 405 nm) was irradiated under the irradiation conditions of each sample shown in Table 13 from ascending order of each sample to culture each sample for 48 hours in a constant temperature incubator set to 35 °C, 8 hours after irradiation start, and the colony number on the medium surface of each sample was counted. As the light source of LED light, a UFL-501-08 UV-UT (manufactured by U-Technology Co., Ltd.) was used. It should be noted that sample 3 which is a petri dish left for 8 hours without irradiating LED light was defined as a control section.

TABLE 13

| | Irradiation conditions | | | | | Sterilization rate |
|---|---|---|---|---|---|---|
| | | Lighting time/ | Irradiation | Irradiation | Colony | relative to control |
| Sample | Irradiance | lights-out time | time | quantity | number | section (%) |
| 1 | 200 Wm⁻² | Continuous | 8 h | 1600 Whm⁻² | 1 | 97.9 |
| 2 | 400 Wm⁻² | 1 h/1 h | 8 h | 1600 Whm⁻² | 3 | 93.6 |
| 3 | 0 (control section) | — | — | 0 | 47 | — |

### (Test Results)

Table 13 shows the colony number of each sample, and the sterilization rate relative to control section in the case of continuously irradiating and the case of intermittently irradiating LED light of 1600 Whm⁻² irradiation quantity. The sterilization rate relative to the control section shown in Table 13 is a value obtained by dividing the colony number of Sample 1 or 2 after irradiating LED light by the colony number in the control section of Sample 3. As shown in Table 13, in Sample 3 of the control section, 47 colonies of bacteria or fungi were observed. In contrast, one colony was observed in Sample 1 and three colonies were observed in Sample 2, and by irradiating 1600 Whm⁻² irradiation quantity in both cases of continuous irradiation and intermittent irradiation, the above sterilization rate reached 90% or higher. From this result, it is speculated that, also for sessile organisms that attach to the surface of structures in air such as airborne bacteria and fungi, it is possible to almost completely remove with an irradiation quantity of 1600 Whm⁻² or more.

### (Influence of Light Irradiation to Microorganisms in Air Confirmation Test 2)

By culturing microorganisms dropped onto LB medium, and counting the colony number appearing after the irradiation of LED light at a lower irradiance than the irradiation condition shown in Table 13, the influence from light irradiation on the microorganisms attaching to a structure in air was confirmed.

The LB medium was prepared by the same procedure as the above “Influence of Light Irradiation on Microorganisms in Air Confirmation Test 1”, and in a state with the lids of five petri dishes in which this LB medium is placed left open, they were placed on a counter about 90 cm height above the floor within a room in the vicinity of the exit to outdoors, and left for 8 hours. The lids were closed after 8 hours, the five petri dishes were respectively labeled Samples 4 to 8, LED light (peak wavelength: 405 nm) was irradiated under the irradiation conditions of each sample shown in Table 14 from ascending order of each sample to culture each sample for 48 hours in a constant temperature incubator set to 35 °C, 8 hours after irradiation start, and the colony number on the medium surface of each sample was counted. As the light source of the LED light, a UFL-501-08 UV-UT (manufactured by U-Technology Co., Ltd.) was used. It should be noted that Samples 6 to 8 which are petri dish left for 8 hours without irradiating LED light were defined as a control section.

TABLE 14

| | Irradiation conditions | | | | | Average colony | Sterilization rate relative to |
|---|---|---|---|---|---|---|---|
| | | Lighting time/ | Irradiation | Irradiation | Colony | number of | average colony number of |
| Sample | Irradiance | lights-out time | time | quantity | number | Samples 6 to 8 | control section (%) |
| 4 | 20 Wm⁻² | Continuous | 8 h | 160 Whm⁻² | 5 | — | 66.7 |
| 5 | 40 Wm⁻² | 1 h/1 h | 8 h | 160 Whm⁻² | 5 | — | 66.7 |
| 6 | 0 (control section) | — | — | 0 | 18 | 15 | — |
| 7 | 0 (control section) | — | — | 0 | 13 | | — |
| 8 | 0 (control section) | — | — | 0 | 15 | | — |

### (Test Results)

Table 14 shows the colony number of each sample, and the sterilization rate relative to the average colony number of the control section in the case of continuously irradiating and the case of intermittently LED light of 160 Whm⁻² irradiation quantity. The sterilization rate relative to the average colony number of the control section shown in Table 14 is a value obtained by dividing the colony number of Sample 4 or 5 after irradiating LED light by the average of the colony numbers in the control sections of Samples 6 to 8. As shown in Table 14, in the three control section, 13 to 18 colonies of bacteria or fungi were observed. In contrast, five colonies were observed in each of Samples 4 and 5, and even in the case of setting the irradiation quantity as 160 Whm⁻², the above sterilization rate reached 60% or higher. From this result, it is speculated that, for sessile organisms attaching to the surface of a structure in air such as airborne bacteria and fungi, even with the irradiation quantity of 160 Whm⁻², a high attachment suppression or sessile organism removal effect can be obtained.

1. An attachment suppression and removal method for a structure to which a sessile organism attaches, the method comprising performing suppression of attachment of the sessile organism to a structure or removal of the sessile organism attached to the structure, by irradiating light onto the structure, based on a light irradiation quantity set for every kind of sessile organism.

2. The attachment suppression and removal method according to claim 1, wherein the light irradiation quantity is 160 Whm⁻² or more.

3. The attachment suppression and removal method according to claim 1, wherein the light irradiation quantity is 480 Whm⁻² or more.

4. The attachment suppression and removal method according to claim 1, wherein the light irradiation quantity is 1920 Whm⁻² or more.

5. The attachment suppression and removal method according to claim 1, wherein the light irradiation quantity is 4800 Whm⁻² or more.

6. The attachment suppression and removal method according to claim 1, wherein irradiance of light irradiated based on the light irradiation quantity is 20 Wm⁻² or more.

7. The attachment suppression and removal method according to claim 1, wherein irradiance of light irradiated based on the light irradiation quantity is 24000 Wm⁻² or more.

8. The attachment suppression and removal method according to claim 1, wherein light irradiated based on the light irradiation quantity has a peak in a wavelength region of 400 to 490 nm.

9. The attachment suppression and removal method according to claim 1, wherein light irradiated based on the light irradiation quantity has a peak in a wavelength region of 400 to 430 nm.
